(19) United States
(12) Patent Application Publication
(10) Pub. No.: US 2016/0022879 A1
(43) Pub. Date: Jan. 28, 2016

(54) HYPOALLERGENIC ORTHOPEDIC SURGICAL INSTRUMENTS AND METHODS

(71) Applicant: Jared Ruben Hillel FORAN, Denver, CO (US)

(72) Inventor: Jared Ruben Hillel FORAN, Denver, CO (US)

(21) Appl. No.: 14/807,211

(22) Filed: Jul. 23, 2015

Related U.S. Application Data
(60) Provisional application No. 62/028,471, filed on Jul. 24, 2014.

(51) Int. Cl. A61L 31/08; C23C 14/35; C25D 5/36; A61B 17/68

(52) U.S. Cl. CPC A61L 31/088; A61B 17/68; C23C 14/35; C25D 5/36; A61B 2017/00964

(57) ABSTRACT

According to an exemplary embodiment, a hypoallergenic orthopedic surgical instrument to prepare bone for the implantation of orthopedic prosthesis may be provided. The instrument may include a hypoallergenic metal alloy such as, but not limited to, titanium nitride, oxidized zirconium, silver, copper, zinc and palladium. In a further embodiment, the hypoallergenic orthopedic surgical may include a high strength, solid low carbon martensitic stainless steel and a thin, high-density hypoallergenic metal alloy coating on the surface of the instrument. According to another exemplary embodiment, a process of building a hypoallergenic orthopedic surgical instrument to prepare bone for the implantation of orthopedic prosthesis may be provided. The process may include cleaning the surface of a high strength, solid low carbon martensitic stainless steel orthopedic surgical instrument to prepare bone for the implantation of orthopedic prosthesis and forming a thin, high-density coating on the surface of the instrument.

### PRIORITY CLAIM

This application claims priority to U.S. Provisional Application No. 62/028,471, filed Jul. 24, 2014, the contents of which are herein incorporated by reference.

### BACKGROUND

Orthopedic surgical instruments and devices, such as saw blades, may provide the ability to cut and prepare bony surfaces for arthroplasty implants. However, the orthopedic surgical instruments used to prepare bone for the implantation of orthopedic prosthesis (such as total hip or knee arthroplasty) may generate substantial metallic debris. These debris, which may often be in the form of very small particles (such as metal filings/shavings), may be abraded from the metal saws, drills, burrs, pins, and screws which may be used in the implantation process. These allergenic particles become lodged in the soft tissues, and may be difficult, if not impossible to completely remove from these tissues. Additionally, these very small particles may have a high surface area and thus may be as, or more, bioavailable than the metals in the prosthesis themselves and may cause reactions or even arthroplasty failure in patients who have allergies or are sensitive to particular metals. This is particularly important for patients who may have, known or, suspected metal allergies.

### SUMMARY

According to an exemplary embodiment, a hypoallergenic orthopedic surgical instrument to prepare bone for the implantation of orthopedic prosthesis may be provided. The instrument may include a hypoallergenic metal alloy such as, but not limited to, titanium nitride, oxidized zirconium, silver, copper, zinc and palladium. In a further embodiment, the hypoallergenic orthopedic surgical instrument may include a high strength, solid low carbon martensitic stainless steel and a thin, high-density hypoallergenic metal alloy coating on the surface of the instrument.

According to another exemplary embodiment, a process of building a hypoallergenic orthopedic surgical instrument to prepare bone for the implantation of orthopedic prosthesis may be provided. The process may include cleaning the surface of a high strength, solid low carbon martensitic stainless steel orthopedic surgical instrument to prepare bone for the implantation of orthopedic prosthesis and forming a thin, high-density coating on the surface of said instrument.

### BRIEF DESCRIPTION OF THE DRAWINGS

Advantages of embodiments of the present invention will be apparent from the following detailed description of the exemplary embodiments. The following detailed description should be considered in conjunction with the accompanying Figures in which:

FIG. 1A may show an exemplary embodiment of hypoallergenic orthopedic surgical power saw blade.

FIG. 1B may show an exemplary embodiment of hypoallergenic orthopedic surgical power saw blade.

FIG. 1C may show an exemplary embodiment of hypoallergenic orthopedic surgical power saw blade.

FIG. 2 may show an exemplary embodiment of hypoallergenic orthopedic drill bit.

FIG. 3 may show an exemplary embodiment of a top perspective view of a hypoallergenic orthopedic surgical oscillating saw blade.

FIG. 4 may show an exemplary embodiment of a hypoallergenic orthopedic cutting block.

FIG. 5A may show an exemplary embodiment of a hypoallergenic orthopedic pin.

FIG. 5B may show an exemplary embodiment of a hypoallergenic orthopedic screw.

FIG. 6A may show an exemplary embodiment of a cross section of a hypoallergenic orthopedic instrument.

FIG. 6B may show another exemplary embodiment of a cross section of hypoallergenic orthopedic instrument.

### DETAILED DESCRIPTION

Aspects of the present invention are disclosed in the following description and related Figures directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

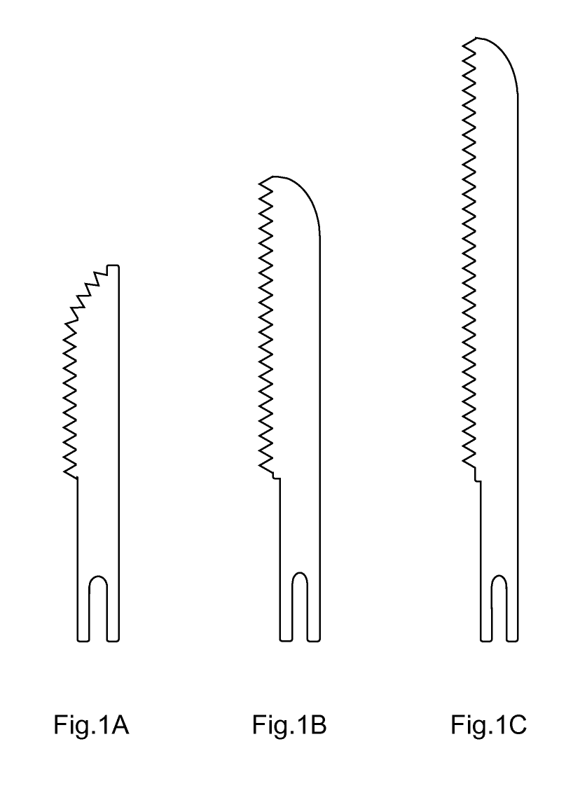

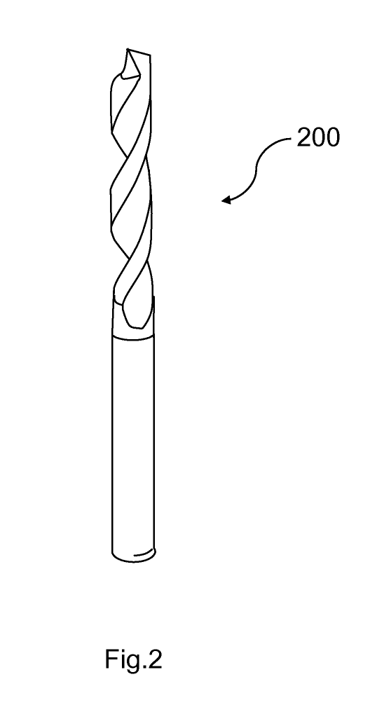

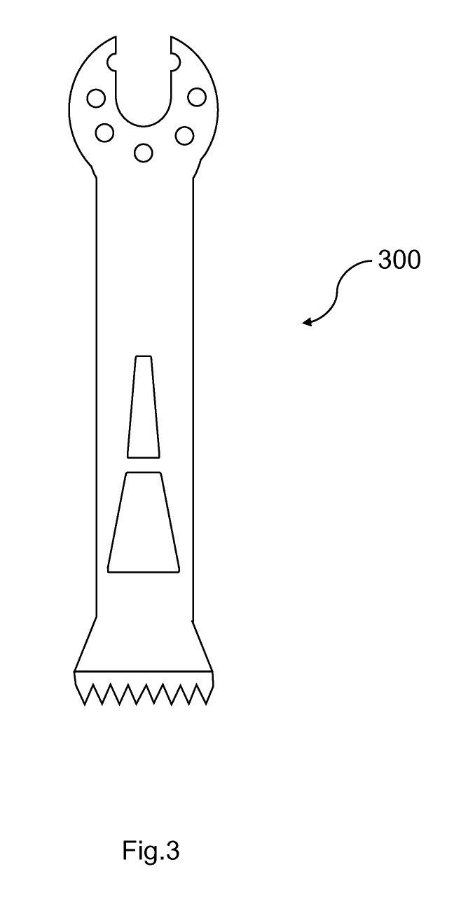

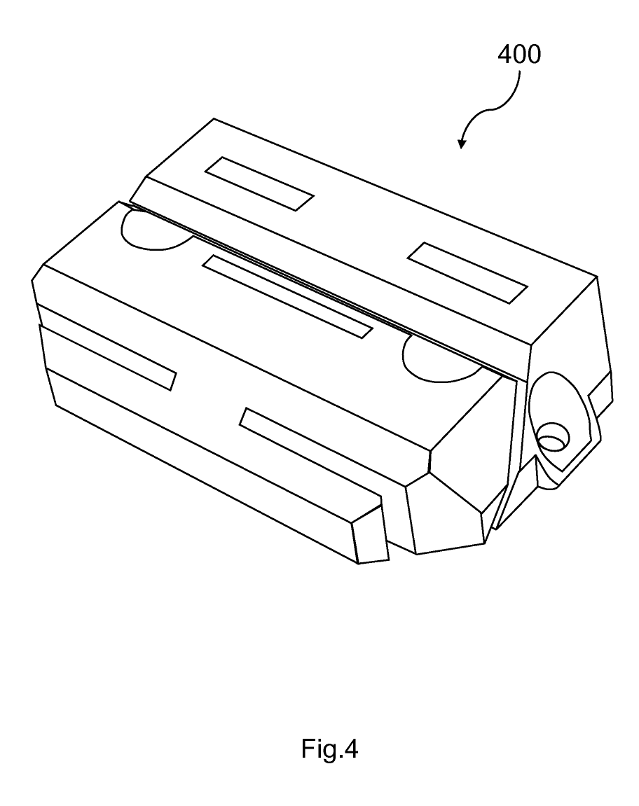

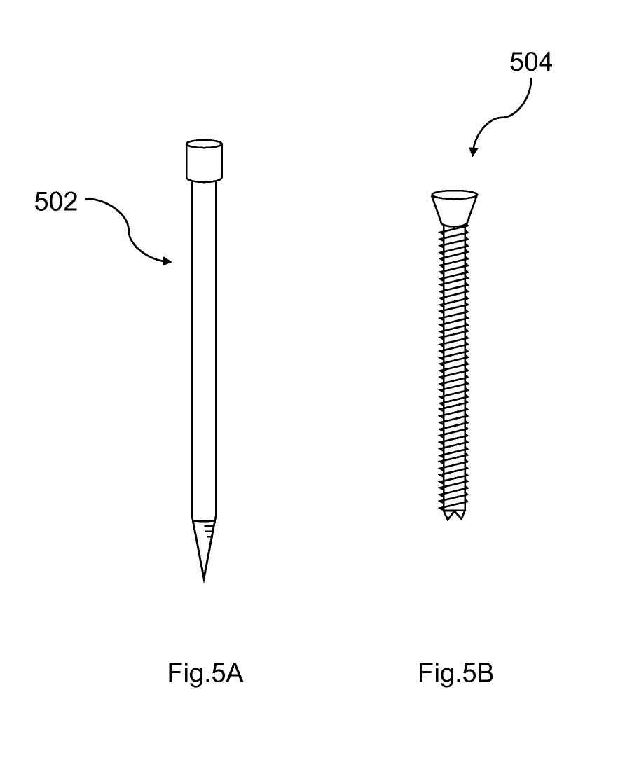

In an exemplary embodiment, a hypoallergenic surgical instrument may include instruments and devices such as, but not limited to, reciprocating power saws blade (FIG. 1A-1C), drill bits (FIG. 2), oscillating saws blade 300 (FIG. 3), cutting guides, blocks, jigs 400 (FIG. 4), orthopedic pins 502 and screws 504 (FIG. 5A-5B), or the like. The hypoallergenic surgical instrument may be entirely made or composed entirely of hypoallergenic material, or may be surface coated with hypoallergenic materials. In an exemplary embodiment, the hypoallergenic material may be have a hardness and material properties that may be compatible with bone preparing tasks. In a further exemplary embodiment, the hypoallergenic materials may include titanium nitride, titanium oxide, and oxidized zirconium (zirconium oxide). It may further be appreciated that any desired material may be utilized as would be understood by a person of ordinary skill in the art.

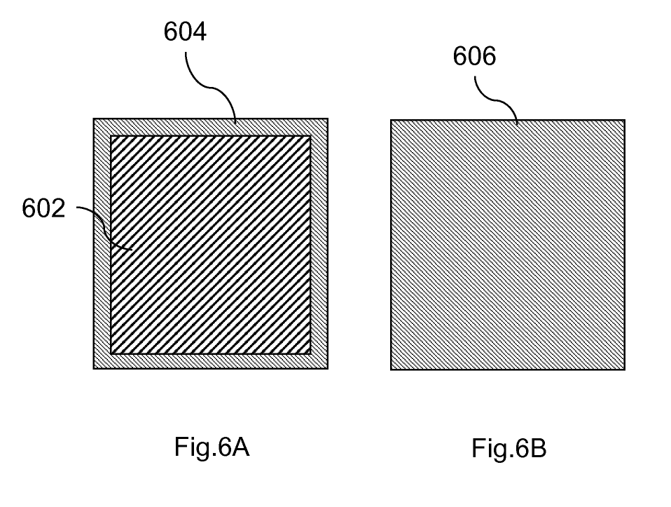

In an exemplary embodiment illustrated in FIG. 6A, the hypoallergenic surgical instrument may be made of solid low-carbon martensitic stainless steel 602 with a high-density coating 604 on the surface. The coating may have a thickness of about 0.5 micrometer to about 20 micrometer. However, it may be appreciated that the thickness of the coating may have any desired value. In an additional embodiment, the coating may be formed using a number of coating techniques including, but not limited to, cathodic arc plasma deposition, magnetron sputtering, electrolytic deposition, or the like. It may be appreciated that the coating technique may include any technique available to a person skilled in the art.

Now referring to FIG. 6B, the hypoallergenic surgical instrument may be entirely made or composed entirely of a solid hypoallergenic material 606.

In a further exemplary embodiment, the high-density coating may be a number of materials, including but not limited to titanium nitride, zirconium oxide, silver, copper, zinc, palladium, or the like.

In an exemplary embodiment, a hypoallergenic surgical instrument may be used to prepare bone on patients who have confirmed metal allergies and are undergoing a surgical procedure including, but not limited to, primary revision joint arthroplasty, revision joint arthroplasty, knee arthroplasty, hip arthroplasty, shoulder arthroplasty, ankle arthroplasty, or the like. The saw blades, drills, cutting guides, pins and screws may have various sizes, dimensions, and shapes, and may be compatible with currently available orthopedic instruments (such as saws, drills, and arthroplasty systems).

The foregoing description and accompanying Figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A hypoallergenic orthopedic surgical instrument to prepare bone for the implantation of orthopedic prosthesis comprising:
an orthopedic surgical instrument composed of a hypoallergenic metal alloy.

2. The hypoallergenic surgical instrument of claim 1, wherein the hypoallergenic metal alloy includes metal and at least one of titanium nitride, oxidized zirconium, silver, copper, zinc and palladium.

3. The hypoallergenic surgical instrument of claim 1, wherein the hypoallergenic metal is titanium nitride.

4. The hypoallergenic surgical instrument of claim 1, wherein the hypoallergenic metal is zirconium oxide.

5. A hypoallergenic orthopedic surgical instrument to prepare bone for the implantation of orthopedic prosthesis comprising:
a high strength, solid low carbon martensitic stainless steel; and
a thin, high-density coating on the surface of said instrument.

6. The hypoallergenic surgical instrument defined in claim 5, wherein the high-density coating is from 1 micrometer to 10 micrometer thick.

7. The hypoallergenic surgical instrument defined in claim 6 wherein the high-density coating is from 1 micrometer to 3 micrometer thick

8. The hypoallergenic surgical instrument defined in claim 5 wherein the high-density coating includes metal and at least one of titanium nitride, zirconium oxide, silver, copper, zinc and palladium.

9. The hypoallergenic surgical instrument defined in claim 5 wherein the high-density coating is titanium nitride.

10. The hypoallergenic surgical instrument defined in claim 5 wherein the high-density coating is zirconium oxide.

11. The hypoallergenic orthopedic surgical instrument defined in claim 5, wherein the high-density coating is applied by cathodic arc plasma deposition.

12. The hypoallergenic orthopedic surgical instrument defined in claim 5, wherein the high-density coating is applied by magnetron sputtering.

13. The hypoallergenic orthopedic surgical instrument defined in claim 5, wherein the high-density coating is applied by electrolytic deposition.

14. A process of building a hypoallergenic orthopedic surgical instrument to prepare bone for the implantation of orthopedic prosthesis comprising:
cleaning the surface of a high strength, solid low carbon martensitic stainless steel orthopedic surgical instrument to prepare bone for the implantation of orthopedic prosthesis; and
forming a thin, high-density coating on the surface of said instrument.

15. The process of claim 12, wherein the high-density coating includes metal and at least one of titanium nitride, zirconium oxide, silver, copper, zinc and palladium.

16. The process of claim 12, wherein the high-density coating is from 1 micrometer to 10 micrometer thick.

17. The process of claim 16, wherein the high-density coating is from 1 micrometer to 3 micrometer thick

18. The process of claim 17, wherein the high-density coating is applied by cathodic arc plasma deposition.

19. The process of claim 17, wherein the high-density coating is applied by magnetron sputtering.

20. The process of claim 17, wherein the high-density coating is applied by electrolytic deposition.
